(19) United States
(12) Patent Application Publication
(10) Pub. No.: US 2024/0361312 A1
(43) Pub. Date: Oct. 31, 2024

(54) METHOD FOR EVALUATING SEVERITY OR PROGNOSIS OF MIXED CONNECTIVE TISSUE DISEASE

(71) Applicant: UNIVERSITY OF OCCUPATIONAL AND ENVIRONMENTAL HEALTH, JAPAN, Kitakyushu (JP)

(72) Inventors: Yoshiya TANAKA, Kitakyushu (JP); Satoshi KUBO, Kitakyushu (JP); Minoru SATOH, Kitakyushu (JP)

(73) Assignee: UNIVERSITY OF OCCUPATIONAL AND ENVIRONMENTAL HEALTH, JAPAN, Kitakyushu (JP)

(21) Appl. No.: 18/432,672

(22) Filed: Feb. 5, 2024

(30) Foreign Application Priority Data
Feb. 6, 2023 (JP) 2023-016268

(51) Int. Cl. G01N 33/564; A61K 31/57

(52) U.S. Cl. CPC G01N 33/564; A61K 31/57; G01N 2800/10; G01N 2800/104; G01N 2800/52

(57) ABSTRACT

The present invention provides a method for evaluating severity or prognosis of a patient with mixed connective tissue disease, which includes
1) a step of bringing a biological sample separated from a patient with mixed connective tissue disease into contact with a carrier in which an antigen containing one or more of component proteins of survival motor neuron complex or an antigenic partial peptide thereof is immobilized on a solid phase to form a complex of an autoantibody in the biological sample and the antigen immobilized on the solid phase,
2) a step of detecting the autoantibody in the complex of the autoantibody and the immobilized antigen, and
3) a step of associating the patient with a severe mixed connective tissue disease or a poor prognosis when the autoantibody is detected from the biological sample separated from the patient in step 2).

### CROSS-REFERENCE TO RELATED APPLICATIONS

This application is based on a patent application No. 2023-016268 filed in Japan (filing date: Feb. 6, 2023), the contents of which are incorporated in full herein.

### TECHNICAL FIELD

The present invention relates to a method of evaluating severity or prognosis of mixed connective tissue disease.

### BACKGROUND ART

In autoimmune diseases, detection of autoantibodies characteristic to each disease relates to diagnosis, organ involvement or prognosis and is of high clinical significance in terms of grouping subset of diseases and application to personalized medicine.

Mixed connective tissue disease (MCTD) is an autoimmune disease characterized by overlapping clinical features of systemic lupus erythematosus (hereinafter sometimes referred to as SLE), systemic sclerosis (hereinafter sometimes referred to as SSc) and idiopathic inflammatory myopathies (hereinafter sometimes referred to as IIM). MCTD is known to be complicated by systemic organ involvement, and notably pulmonary arterial hypertension (PAH) and interstitial lung disease (ILD) affect prognosis of life (Non-patent document 1: Rheumatology (Oxford). 2013; 52 (7): 1208-13, Non-patent document 2: Arthritis Rheum. 1999; 42 (5): 899-909, Non-patent document 3: Ann Rheum Dis. 2012; 71 (12): 1966-72, Non-patent document 4: Clin Exp Rheumatol. 2018; 36 (4): 648-651, Non-patent document 5: Rheumatology (Oxford). 2018; 57 (2): 255-62).

Anti-U1 ribonucleoprotein (U1 RNP) antibodies are an autoantibody of high diagnostic significance in MCTD. Anti-U1 RNP antibodies are essential for the diagnosis of MCTD and sensitivity to the diagnosis of MCTD is 100%. However, anti-U1 RNP antibodies can be positive in other diseases, such as SLE and SSc; thus, their specificity and positive predictive value for MCTD diagnosis are not high. In addition, association with organ involvement characteristic of MCTD, such as PAH and ILD; disease activity and prognosis is poor.

The present inventors have conducted autoantibody analysis using immunoprecipitation on patients with autoimmune diseases. In the preliminary studies, the present inventors have reported that anti-Survival of Motor Neuron (hereinafter sometimes referred to as SMN) complex antibodies were detected in several patients with IIM/SSc overlap syndrome (Non-patent document 6: Arthritis Rheum. 2011; 63 (7): 1972-8, Non-patent document 7: Clin Rev Allergy Immunol. 2012; 42 (1): 16-25), and around 25% of the cases positive for anti-U1 RNP antibodies also tested positive for anti-SMN complex antibodies (Non-patent document 8; Rheumatology (Oxford). 2018; 57 (1): 199-200). However, the significance of anti-SMN complex antibodies in MCTD is unclear.

### SUMMARY OF INVENTION

### Technical Problem

The present invention aims to provide a new biomarker for evaluating severity and prognosis of MCTD, and a method for evaluating severity and prognosis of MCTD using the biomarker.

### Solution to Problem

The present inventors have evaluated the clinical significance of anti-SMN complex autoantibodies in 158 consecutively hospitalized patients who were positive for anti-U1 RNP autoantibodies, encompassing those with MCTD and those with SLE or SSc who were not diagnosed with MCTD. As a result, they found that anti-SMN complex autoantibodies are rarely detected in non-MCTD patients, show high specificity, and may serve as an adjunctive biomarker for diagnosis of MCTD. Anti-SMN complex autoantibodies-positive MCTD patients had higher prevalence of complication with PAH and ILD and had a poorer prognosis than negative patients. Anti-SMN complex antibodies were associated with “typical MCTD” that had clinical features characteristic of MCTD. Based on these findings, the present inventors have investigated further and completed the present invention.

Accordingly, the present invention relates to the followings.
[1] A method for evaluating severity of a patient with mixed connective tissue disease, which comprises
   1) a step of bringing a biological sample separated from a patient with mixed connective tissue disease into contact with a carrier in which an antigen containing one or more of a component protein of survival motor neuron complex or an antigenic partial peptide thereof is immobilized on a solid phase to form a complex of an autoantibody in the biological sample and the antigen immobilized on the solid phase,
   2) a step of detecting the autoantibody in the complex of the autoantibody and the immobilized antigen, and
   3) a step of associating the patient with a severe mixed connective tissue disease when the autoantibody is detected from the biological sample separated from the patient in step 2).
[2] The method of [1], wherein the component protein of the survival motor neuron complex is selected from the group consisting of SMN, Gemin 3, Gemin 4 and Gemin 5.
[3] The method of [1] or [2], wherein the detection of the autoantibody is performed by bringing an anti-human IgG antibody or a functional binding fragment thereof into contact with the complex of the autoantibody and the immobilized antigen.
[4] The method of any of [1] to [3], wherein the severe mixed connective tissue disease is a mixed connective tissue disease complicated by pulmonary arterial hypertension or interstitial lung disease.
[5] The method of any of [1] to [4], wherein the severe mixed connective tissue disease is a mixed connective tissue disease accompanied by all of systemic lupus erythematosus-like, systemic sclerosis-like, and idiopathic inflammatory myopathy-like clinical symptoms.
[6] A method for evaluating prognosis of a patient with mixed connective tissue disease, which comprises
   1) a step of bringing a biological sample separated from a patient with mixed connective tissue disease into contact with a carrier in which an antigen containing one or more of component proteins of survival motor neuron complex or an antigenic partial peptide thereof is immobilized on a solid phase to form a complex of an autoantibody in the biological sample and the antigen immobilized on the solid phase,
   2) a step of detecting the autoantibody in the complex of the autoantibody and the immobilized antigen, and
   3) a step of associating the patient with a mixed connective tissue disease with a poor prognosis when the autoantibody is detected from the biological sample separated from the patient in step 2).
[7] The method of [6], wherein the component protein of the survival motor neuron complex is selected from the group consisting of SMN, Gemin 3, Gemin 4 and Gemin 5.
[8] The method of [6] or [7], wherein the detection of the autoantibody is performed by bringing an anti-human IgG antibody or a functional binding fragment thereof into contact with the complex of the autoantibody and the immobilized antigen.
[9] A method of treating mixed connective tissue disease, which comprises detecting an autoantibody to survival motor neuron complex or a component protein of the survival motor neuron complex in a biological sample separated from a patient with mixed connective tissue disease, and administering glucocorticoid to the patient with the mixed connective tissue disease when the patient is positive for the autoantibody against the survival motor neuron complex or the component protein of the survival motor neuron complex.
The method of [9], wherein the detection of an autoantibody against survival motor neuron complex or a component protein of the survival motor neuron complex is performed by a method comprising
   1) a step of bringing a biological sample separated from a patient with mixed connective tissue disease into contact with a carrier in which an antigen containing one or more of component proteins of the survival motor neuron complex or an antigenic partial peptide thereof is immobilized on a solid phase to form a complex of an autoantibody in the biological sample and the antigen immobilized on the solid phase, and
   2) a step of detecting the autoantibody in the complex of the autoantibody and the immobilized antigen.
[11] The method of [10], wherein the component protein of the survival motor neuron complex is selected from the group consisting of SMN, Gemin 3, Gemin 4 and Gemin 5.
[12] The method of or [11], wherein the detection of the autoantibody is performed by bringing an anti-human IgG antibody or a functional binding fragment thereof into contact with the complex of the autoantibody and the immobilized antigen.
[1a] A method for evaluating severity of mixed connective tissue disease, which comprises detecting an anti-survival motor neuron complex autoantibody in a biological sample separated from a patient with mixed connective tissue disease.
[2a] The method of [1a], wherein the patient is associated with severe mixed connective tissue disease when the patient is positive for anti-survival motor neuron complex autoantibody.
[3a] The method of [2a], wherein the severe mixed connective tissue disease is a mixed connective tissue disease complicated by pulmonary arterial hypertension or interstitial lung disease.
[4a] The method of [2a], wherein the severe mixed connective tissue disease is a mixed connective tissue disease accompanied by all of systemic lupus erythematosus-like, systemic sclerosis-like, and idiopathic inflammatory myopathy-like clinical symptoms.
[5a] A method for evaluating prognosis of mixed connective tissue disease, which comprises detecting an anti-survival motor neuron complex autoantibody in a biological sample separated from a patient with mixed connective tissue disease.
[6a] The method of [5a], wherein the patient is associated with a poor prognosis when the patient is positive for anti-survival motor neuron complex autoantibody.
[7a] A kit for evaluating severity of mixed connective tissue disease, which comprises survival motor neuron complex.
[8a] A kit for evaluating prognosis of mixed connective tissue disease, which comprises survival motor neuron complex.
[9a] A biomarker for evaluating severity of mixed connective tissue disease, which comprises an anti-survival motor neuron complex autoantibody.
[10a] A biomarker for evaluating prognosis of mixed connective tissue disease, which comprises an anti-survival motor neuron complex autoantibody.

### Effects of Invention

The present invention provides a new biomarker for evaluating severity or prognosis of MCTD, and makes it possible to evaluate severity, organ involvement, disease activity, and prognosis of MCTD. Although the severity classification is used for the medical expense benefit for Ministry of Health, Labour and Welfare (MHLW)-designated intractable diseases, it is composed mostly of subjective items. The present invention enables severity classification of MCTD based on objective indexes.

### BRIEF DESCRIPTION OF DRAWINGS

FIG. 1 shows the analysis results of anti-SMN complex reactivity of sera from MCTD patients by immunoprecipitation. Immunoprecipitates using ³⁵S-methionine/cysteine-labeled K562 cell extract was analyzed by 12.5% (A) and 8.0% (B) sodium dodecyl sulfate-polyacrylamide gel electrophoresis. Lanes 1-9 contain sera from the patients with MCTD. The positions of the components of the SMN complex (SMN, Gemin 2, Gemin 3/4, Gemin 5, Gemin 6/7) as well as the components of small nuclear RNPs (U1-70 k, U1-A, U1-C) and Sm core particles (B′/B, D1/D2/D3, E/F, and G) are indicated at the left. SMN: survival motor neuron; mab: mouse monoclonal anti-SMN antibody 2B1; hu: human prototype anti-SMN antibodies positive serum; NHS: normal human serum.

FIG. 2 shows the prevalence of various autoantibodies among patients with MCTD, SLE and SSc. P-values are for MCTD vs SLE and SSc, determined by Fisher's exact test. * p<0.05, ** p<0.01, and *** p<0.001. MCTD: mixed connective tissue disease, SLE: systemic lupus erythematosus, SSc: systemic sclerosis, SMN: survival motor neuron.

FIG. 3A shows the relationship between the presence or absence of anti-SMN complex antibodies (Anti-SMN) and clinical/laboratory findings (characteristic organ involvements) in MCTD patients (n=67). Data are median (interquartile range) unless otherwise stated. P-values were determined by Fisher's exact test, t-test or Mann-Whitney test. * p<0.05, ** p<0.01, *** p<0.001.

FIG. 3B shows the relationship between the presence or absence of anti-SMN complex antibodies (Anti-SMN) and clinical/laboratory findings (nailfold video capillaroscopy) in MCTD patients (n=67). Data are median (interquartile range) unless otherwise stated. P-values were determined by Fisher's exact test, t-test or Mann-Whitney test. * p<0.05, ** p<0.01, *** p<0.001.

FIG. 3C shows the relationship between the presence or absence of anti-SMN complex antibodies (Anti-SMN) and clinical/laboratory findings (overlapping manifestations) in MCTD patients (n=67). Data are median (interquartile range) unless otherwise stated. P-values were determined by Fisher's exact test, t-test or Mann-Whitney test. * p<0.05, ** p<0.01, *** p<0.001.

FIG. 3D shows the relationship between the presence or absence of anti-SMN complex antibodies (Anti-SMN) and clinical/laboratory findings (other auto antibodies) in MCTD patients (n=67). Data are median (interquartile range) unless otherwise stated. P-values were determined by Fisher's exact test, t-test or Mann-Whitney test. * p<0.05, ** p<0.01, *** p<0.001.

FIG. 4 shows the relationship between anti-SMN complex antibodies and combination of overlapping manifestations.

FIG. 5A shows a comparison of the percentage of high-dose glucocorticoid therapy used in initial treatment in MCTD patients with and without anti-SMN complex antibodies.

FIG. 5B shows a comparison of 1-year survival rates in MCTD patients with and without anti-SMN complex antibodies.

FIG. 6 shows a relationship between anti-SMN complex antibodies and PAH/ILD in MCTD patients.

### DETAILED DESCRIPTION OF THE INVENTION

The present invention provides a method for evaluating severity or prognosis of mixed connective tissue disease, which comprises detecting an anti-SMN complex autoantibody in a biological sample separated from a patient with mixed connective tissue disease (hereinafter referred to as the method of the present invention).

Mixed connective tissue disease (MCTD) is an autoimmune disease characterized by overlapping 2 or more of systemic lupus erythematosus (SLE)-like, systemic sclerosis (SSc)-like and idiopathic inflammatory myopathies (IIM)-like clinical symptoms and being positive for anti-U1 RNP autoantibody in blood test. MCTD can be diagnosed based on the MCTD Diagnostic Criteria 2019 Revision (Mod Rheumatol. 2021; 31 (1): 29-33) published by the Japanese Research Group of the Ministry of Health, Labor and Welfare.

The survival motor neuron (SMN) complex is a protein complex involved in formation of small nuclear ribonucleoprotein (snRNP) and RNA metabolism and splicing in eukaryotic cells (Trends Mol.

Med., 2006; 12 (3): 13-21, Semin Cell Dev Biol, 2014; 32:22-9). The SMN complex is composed of SMN and Gemin proteins (Gemins 2-7). The most representative components constituting the SMN complex are SMN, Gemin 2 (also called SIP1), Gemin 3 (also called DP103, Ddx20) and Gemin 4 (also called GIP1). The SMN complex contains SMN and one or a plurality of Gemin proteins selected from Gemins 2-7. The SMN complex recognized by the anti-SMN complex antibody to be detected in the present invention preferably contains SMN, Gemin 3 and Gemin 4 as components, and may or may not contain other components (for example, Gemin 5 or the like).

The anti-SMN complex autoantibody refers to an autoantibody that specifically recognizes the SMN complex and includes an autoantibody that specifically recognizes any component within the SMN complex and an autoantibody that specifically recognizes the three-dimensional structure of the SMN complex formed by assembly of multiple components. The anti-SMN complex antibodies to be detected in the present invention include an autoantibody that specifically recognizes SMN in the SMN complex, an autoantibody that specifically recognizes Gemin 3 in the SMN complex, an autoantibody that specifically recognizes Gemin 4 in the SMN complex, an autoantibody that specifically recognizes Gemin 5 in the SMN complex, an autoantibody that specifically recognizes a three-dimensional structure of the SMN complex containing SMN, Gemin 3 and Gemin 4 or the SMN complex containing SMN, Gemin 3, Gemin 4 and Gemin 5.

“Specific” means that an antibody which binds specifically to one or a plurality of binding partners does not exhibit any significant binding to molecules other than those partners. Furthermore, “specific” is also used when the antigen binding site is specific for a particular epitope among multiple epitopes in an antigen. When the epitope to which the antigen binding site binds is contained in a plurality of different antigens, an antibody containing the antigen binding site may bind to a variety of antigens having that epitope.

The biological samples used in the method of the present invention are not particularly limited as long as they are collected from MCTD patients and may contain the anti-SMN complex autoantibody to be detected. Examples of the biological samples include blood, plasma, serum, lymph fluid, spinal fluid, ascites fluid, joint fluid, urine, sweat, saliva, and other body fluids or their fractions, and preferably blood, plasma, or serum.

Detection of anti-SMN complex autoantibodies in biological samples can be performed by an immunological assay (e.g., immunoprecipitation, ELISA, FIA, RIA, Western blot, etc.) using cell lysate containing the SMN complex or isolated and purified SMN complex.

Detection of anti-SMN complex autoantibodies in a biological sample can also be performed by immunological assay (e.g., immunoprecipitation, ELISA, FIA, RIA, Western blot, etc.) using a component protein of the SMN complex (SMN, Gemin 3, Gemin 4, Gemin 5 or the like) or an antigenic partial peptide thereof. The component protein of the SMN complex or an antigenic partial peptide thereof to be used for detection is preferably isolated or purified. The purity of the isolated or purified protein or peptide (the ratio of the weight of the intended specific protein or peptide to the weight of the total proteins or peptides) is usually 50% or more, preferably 70% or more, more preferably 90% or more, and most preferably 95% or more (e.g., 100%). In one embodiment, the component protein of the SMN complex or an antigenic partial peptide thereof is used for detection in a form not involved in the SMN complex (i.e., in a released form).

A tag peptide may be added to the component protein of the SMN complex or an antigenic partial peptide thereof to be used for detection. A tag peptide refers to a peptide that is added to a protein to facilitate detection or purification of the protein. Examples of the tag peptides include, but are not limited to, epitope tags, fluorescent proteins, immunoglobulin Fc region or the like. Epitope tag refers to a peptide that is specifically recognized by an antibody or other binding partners, and includes Flag, polyhistidine, c-Myc tag, HA, AU1, GST, MBP or the like.

The component protein of the SMN complex or an antigenic partial peptide thereof to be used for detection may be labeled with an appropriate labeling agent (biotin, enzyme, fluorescent substance, luminescent substance or the like).

When applying these individual immunological measurement methods to the method of the present invention, it is unnecessary to set special conditions, procedures or the like. By making ordinary technical considerations for those skilled in the art to the ordinary conditions and procedures in each method, a measurement system for anti-SMN complex autoantibody can be constructed. For details of these general technical means, compendia, books or the like can be referred to. For example, Hiroshi Irie, ed., “Radioimmunoassay” (Kodansha Ltd., published in 1974), Hiroshi Irie, ed., “Sequel to the Radioimmunoassay” (Kodansha Ltd., published in 1979), Eiji Ishikawa et al., ed., “Enzyme Immunoassay” (Igakushoin, published in 1978), Eiji Ishikawa et al., ed., “Enzyme Immunoassay” (2nd ed.) (Igakushoin, published in 1982), Eiji Ishikawa et al., ed., “Enzyme Immunoassay” (3rd ed.) (Igakushoin, published in 1987), “Methods in ENZYMOLOGY” Vol. 70 (Immunochemical Techniques (Part A)), ibid., Vol. 73 (Immunochemical Techniques (Part B)), ibid., Vol. 74 (Immunochemical Techniques (Part C)), ibid., Vol. 84 (Immunochemical Techniques (Part D: Selected Immunoassays)), ibid., Vol. 92 (Immunochemical Techniques (Part E: Monoclonal Antibodies and General Immunoassay Methods)), ibid., Vol. 121 (Immunochemical Techniques (Part I: Hybridoma Technology and Monoclonal Antibodies)) (all published by Academic Press Publishing) may be referred to.

In one specific embodiment, a lysate of human cells (e.g., K562) containing SMN complex is mixed with a biological sample which may contain anti-SMN complex autoantibodies, and the formed immune complex is collected by immunoprecipitation and evaluated to determine whether the immunoprecipitate contains the SMN complex or not. This evaluation is performed, for example, by using a cell lysate labeled with a radioisotope (e.g., 35S-methionine/cysteine), separating the immunoprecipitate by electrophoresis (e.g., SDS-PAGE), and comparing the migration patterns of the immunoprecipitate with those of a positive control sample containing an antibody specifically recognizing the SMN complex to evaluate the presence or absence of bands corresponding to SMN complex components (SMN, Gemin 3, Gemin 4, etc.) (Arthritis Rheum. 2007; 56 (2): 596-604). Alternatively, the presence of each SMN complex components (SMN, Gemin 3, Gemin 4) in the immunoprecipitate is evaluated by an immunological assay (e.g., Western blotting) using antibodies that specifically recognize each component of the SMN complex (SMN, Gemin 3, Gemin 4 or the like).

In another embodiment, the SMN complex is isolated or purified from human cells bearing SMN complexes by an antibody column using an antibody that specifically recognizes any of the components of the SMN complex (SMN, Gemin 3, Gemin 4 or the like) (e.g., anti-SMN antibody). The isolated or purified SMN complex is then brought into contact with the biological sample to be evaluated to determine whether or not the biological sample contains an autoantibody that binds to the isolated or purified SMN complex by an immunological assay (e.g., ELISA, FIA, RIA or the like).

In a further embodiment, a biological sample separated from a patient with mixed connective tissue disease is brought into contact with a carrier in which an antigen containing one or more of component proteins of the SMN complex (SMN, Gemin 3, Gemin 4, Gemin 5 or the like) or an antigenic partial peptide thereof is immobilized on a solid phase to form a complex of an autoantibody in the biological sample and the antigen immobilized on the solid phase, and the autoantibody in the complex of said autoantibody and the immobilized antigen is detected by an immunological assay (e.g., ELISA, FIA, RIA or the like). The content of the component protein of the SMN complex or an antigenic partial peptide thereof in the antigen immobilized on the solid phase (the percentage of the weight of the component protein of the SMN complex or an antigenic partial peptide thereof to the weight of the total antigen proteins or peptides) is usually 50% or more, preferably 70% or more, more preferably 90% or more, and most preferably 95% or more (e.g., 100%). In one embodiment, the antigen to be immobilized on the solid phase consists of a component protein of the SMN complex or an antigenic partial peptide thereof.

Multiple kinds of component proteins of the SMN complex or antigenic partial peptides thereof may be immobilized, or only a single kind of component proteins of the SMN complex or a partial peptide thereof may be immobilized. Preferably, the component protein of the SMN complex is one selected from the group consisting of SMN, Gemin 3, Gemin 4 and Gemin 5, and more preferably selected from the group consisting of SMN, Gemin 3 and Gemin 4. In one embodiment, the component protein of the SMN complex or an antigenic partial peptide thereof contained in the antigen to be immobilized is only 1, 2, 3 or 4 selected from the group consisting of SMN or an antigenic partial peptide thereof, Gemin 3 or an antigenic partial peptide thereof, Gemin 4 or an antigenic partial peptide thereof, and Gemin 5 or an antigenic partial peptide thereof. In another embodiment, the component protein of the SMN complex or an antigenic partial peptide thereof contained in the antigen to be immobilized is only 1, 2 or 3 selected from the group consisting of SMN or an antigenic partial peptide thereof, Gemin 3 or an antigenic partial peptide thereof, and Gemin 4 or an antigenic partial peptide thereof. In one embodiment, the antigen to be immobilized on the solid phase does not contain Gemin 5 or a partial peptide thereof.

Detection of an autoantibody in a complex of the autoantibody and an immobilized antigen can be performed by bringing the antibody against human immunoglobulin (e.g., IgG) or a functional binding fragment thereof into contact with the complex of the autoantibody and the immobilized antigen and detecting the antibody against human immunoglobulin or a functional binding fragment thereof bound to the complex. For example, an antibody against human immunoglobulin or a functional partial fragment is labeled with an appropriate enzyme and reacted with a substrate.

Then in one embodiment, when anti-SMN complex autoantibody is detected in the biological sample (i.e., “positive” case), it is associated with a severe mixed connective tissue disease. Examples of the “severe mixed connective tissue disease” include a mixed connective tissue disease complicated by pulmonary arterial hypertension or interstitial lung disease, and a mixed connective tissue disease accompanied by all of systemic lupus erythematosus-like, systemic sclerosis-like, and idiopathic inflammatory myopathy-like clinical symptoms. Examples of the systemic lupus erythematosus-like clinical symptoms include, but are not limited to, polyarthritis, lymphadenopathy, facial erythema, pericarditis or pleuritis, cytopenia, hypocomplementemia or the like. Examples of the systemic sclerosis-like clinical symptoms include, but are not limited to, skin hardening, interstitial lung disease, high level KL-6 or the like. Examples of the idiopathic inflammatory myopathy-like clinical symptoms include, but are not limited to, muscle weakness, high level serum creatine kinase (CK), and myogenic abnormalities on electromyogram. When the anti-SMN complex autoantibody is positive, it can be evaluated that the mixed connective tissue disease is more likely to be severe, complicated by pulmonary arterial hypertension or interstitial lung disease, or accompanied by all three of systemic lupus erythematosus-like, systemic sclerosis-like, and idiopathic inflammatory myopathy-like clinical symptoms as compared to the negative case.

In another embodiment, when the anti-SMN complex autoantibody is detected in a biological sample (i.e., “positive” case), it is associated with a poor prognosis. That is, when anti-SMN complex autoantibody is positive, it can be evaluated as a poor prognosis and likely to have a lower future survival rate as compared to the negative case.

In addition, when the anti-SMN complex autoantibody is detected in a biological sample (i.e., “positive” case), the patient with mixed connective tissue disease is considered to be severe or have a poor prognosis, and a physician may select glucocorticoid therapy (preferably high-dose glucocorticoid therapy) as a treatment regimen for said patient with the mixed connective tissue disease and administer glucocorticoid (preferably high-dose glucocorticoid) to said patient with the mixed connective tissue disease. Accordingly, by detecting an anti-SMN complex autoantibody in a biological sample, a physician can select an appropriate treatment regimen for the patient with mixed connective tissue disease.

The present invention extends to a kit for evaluating severity or prognosis of mixed connective tissue disease (the kit of the present invention). The kit contains a SMN complex for detecting an anti-SMN complex autoantibody. The definition of “SMN complex” is as described above. The SMN complex contained in the kit of the present invention preferably comprises SMN, Gemin 3 and Gemin 4 as components, and may or may not comprise other components (e.g. Gemin 5 or the like). Being “isolated or purified” means that an operation to remove components other than the components of interest has been applied to the state of natural or synthesized presence. The purity of the isolated or purified SMN complex (percentage of the SMN complex among total proteins ((w/w) %)) is generally 10% or more, preferably 50% or more, more preferably 80% or more, and further preferably 90% or more. In the kit of the present invention, the SMN complex is brought into contact with a biological sample of a patient with mixed connective tissue disease and an anti-SMN complex autoantibodies bound to the SMN complex is detected using an immunological assay.

In a further aspect, the kit of the present invention contains, instead of the SMN complex, one or more of the component proteins of the SMN complex or an antigenic partial peptide thereof. The definition of the component protein of the SMN complex or an antigenic partial peptide thereof is as described above. The component protein of the SMN complex or an antigenic partial peptide thereof contained in the kit of the present invention is preferably isolated or purified. In one embodiment, the component protein of the SMN complex or an antigenic partial peptide thereof may be comprised in the kit of the present invention in a form not involved in the SMN complex (i.e., in a released form). The kit may comprise multiple kinds of component proteins of the SMN complex or antigenic partial peptides thereof, or only a single kind of component proteins of the SMN complex component or a partial peptide thereof. Preferably, the component protein of the SMN complex is one selected from the group consisting of SMN, Gemin 3, Gemin 4 and Gemin 5, and more preferably selected from the group consisting of SMN, Gemin 3 and Gemin 4. In one embodiment, the component protein of the SMN complex or an antigenic partial peptide thereof contained in the kit of the present invention is only 1, 2, 3 or 4 selected from the group consisting of SMN or an antigenic partial peptide thereof, Gemin 3 or an antigenic partial peptide thereof, Gemin 4 or an antigenic partial peptide thereof, and Gemin 5 or an antigenic partial peptide thereof. In another embodiment, the component protein of the SMN complex or an antigenic partial peptide thereof contained in the kit of the present invention is only 1, 2 or 3 selected from the group consisting of SMN or an antigenic partial peptide thereof, Gemin 3 or an antigenic partial peptide thereof, and Gemin 4 or an antigenic partial peptide thereof. In one embodiment, the kit of the present invention does not contain Gemin 5 or a partial peptide thereof.

In the method of the present invention described above, the severity or prognosis of the mixed connective tissue disease can be easily evaluated by detecting anti-SMN complex autoantibodies in a biological sample of a patient with mixed connective tissue disease using the kit of the present invention.

The SMN complex, or the component protein of the SMN complex or an antigenic partial peptide thereof may be immobilized on a solid phase in advance. The solid phase available for the diagnosis kit of the present invention is not particularly limited and includes, for example, polymers such as polystyrene or the like, and insoluble carriers such as glass beads, magnetic particles, microplate, filter paper for immunochromatography, glass filter or the like.

The kit of the present invention may contain other reagents or the like in addition to the SMN complex, or the component protein of the SMN complex or an antigenic partial peptide thereof. These reagents or the like may be combined with the SMN complex, or the component protein of SMN complex or an antigenic partial peptide thereof in advance, or may be contained in separate tubes. Examples of other reagents include a secondary antibody (for example, anti-human IgG or the like labeled with an enzyme such as peroxidase or alkaline phosphatase or fluorescent dye), blocking solution, substrate, solid phase, reaction container as well as treatment liquid, buffer solution for diluting the SMN complex, positive control (i.e., anti-SMN complex specific antibody, human serum containing anti-SMN complex autoantibodies), negative control, instructions describing the protocol or the like. These components may be mixed in advance as needed.

All references cited in the present specification, including publication, patent document and the like, are hereby incorporated individually and specifically by reference, to the extent that the entireties thereof have been specifically disclosed herein.

The present invention is explained in more detail in the following by referring to Examples, which are not to be construed as limitative.

### Examples

### 1. Methods

### (Patients)

This multicenter (University of Occupational and Environmental Health, Japan, Wakamatsu Hospital of the University of Occupational and Environmental Health, Kitakyushu General Hospital, Fukuoka Yutaka Central Hospital, and Tobata General Hospital), and observational study included 158 newly diagnosed and previously untreated consecutive cases with anti-U1 RNP Abs who consented to the participation in the study between April 2014 and August 2022. MCTD patients were diagnosed based on the Diagnostic Criteria for MCTD 2004, issued by the Japanese Research Group of the MHLW for systemic autoimmune diseases (the Kasukawa criteria), between April 2014 and July 2022, and based on the MCTD Diagnostic Criteria 2019 Revision issued by the Japanese Research Group of MHLW in Japan (hereafter, the new diagnostic criteria 2019) (Mod Rheumatol. 2021; 31 (1): 29-33) for January 2020 onwards, respectively. In this study, all patients (n=67) with MCTD were confirmed to fulfill the new diagnostic criteria 2019, which both sensitivity (90.6%) and specificity (98.4%) are higher than those of other existing diagnostic criteria (Mod Rheumatol. 2021; 31 (1): 29-33). In addition, this study also included the following anti-U1 RNP antibodies positive controls who were diagnosed during the same period: 74 patients with SLE who fulfilled the 2012 Systemic Lupus International Collaborating Clinics Classification (SLICC) criteria (Arthritis Rheum. 2012; 64 (8): 2677-86), 17 patients with SSc who fulfilled the American College of Rheumatology (ACR)/European League Against Rheumatism (EULAR) 2013 classification criteria (Ann Rheum Dis. 2013; 72 (11): 1747-55). In this study, there was no patient with IIM who fulfilled the ACR/EULAR 2017 classification criteria (Ann Rheum Dis. 2017; 76 (12): 1955-64). The patients who were classified as having overlapping diagnosis of SLE, SSc and IIM were excluded. This study was approved by the ethics review board of the University of Occupational and Environmental Health (UMIN ID 000014293; based on a study on rheumatic connective tissue diseases with the objectives of performing quantitative evaluation using NVC, cell surface antigens, autoantibodies, and skin biopsy and elucidating the pathology [SCORPION study]). A signed informed consent was obtained from all subjects in accordance with the Declaration of Helsinki and its subsequent modifications.

### (Clinical Evaluation)

In this study, disease duration was defined as time from the first non-Raynaud's phenomenon manifestation. The clinical and laboratory parameters selected for evaluation were mainly those included in the new MCTD diagnostic criteria 2019. The occurrence of Raynaud's phenomenon was evaluated as a common manifestation, and the presence or absence of PAH and aseptic meningitis was evaluated as characteristic organ involvement. With regard to overlapping manifestations, SLE-like manifestations included polyarthritis, lymphadenopathy, facial erythema, pericarditis or pleuritis, cytopenia (leukocytes≤4000/μL or thrombocytes≤1.0×105/μL), hypocomplementemia (CH50<30 U/ml). SSc-like manifestations included skin hardening (sclerodactyly and high modified Rodnan skin score >4 points), interstitial lung disease (ILD), and high serum levels of Krebs von den Lungen-6 (KL-6, >500U/ml). For the assessment of ILD, high-resolution computed tomography (HRCT) was used in all patients. ILD was diagnosed by the existence of interstitial opacities on HRCT image. IIM-like manifestations included muscle weakness (Manual Muscle Testing 4 or under out of 5), serum creatine kinase (CK) levels, and myogenic abnormalities on electromyogram (EMG). The other parameters included fever (≥37.5° C.), the presence or absence of nephritis, the levels of C-reactive protein (CRP), erythrocyte sedimentation rate (ESR), and immunoglobulin G (IgG). Nailfold videocapillaroscopy (NVC) scleroderma patterns were defined based on the “Fast Track algorithm”, the standardized description of capillaroscopic characteristics, proposed by the EULAR Study Group on Microcirculation in Rheumatic Diseases (Autoimmun Rev. 2020; 19 (3): 102458, Nat Rev Rheumatol. 2021; 17 (11): 665-77, Rheumatology (Oxford). 2022; 61 (12): 4875-4884). Based on the proposals of the 6th World Symposium on Pulmonary Hypertension (Eur Respir J. 2019; 53 (1)), PAH was diagnosed based on mean pulmonary artery pressure of >20 mmHg, pulmonary capillary wedge pressure of <15 mmHg, and pulmonary vascular resistance >3 wd units, as measured by right heart catheterization.

### (Immunoprecipitation)

Autoantibodies in sera were screened by immunoprecipitation using ³⁵S-methionine/cysteine labeled K562 cell extract (Arthritis Rheum. 2007; 56 (2): 596-604). Samples were analyzed on 12.5% and 8% acrylamide gels to fractionate components of snRNPs and SMN complex (Cell. 1995; 80 (1): 155-65, Satoh et al., Antihistone and antispliceosome antibodies, Systemic Lupus Erythematosus, 2016: p.213-221). Specificity of the autoantibodies was determined using previously described reference sera. The presence of antibodies to SMN complex was defined by the presence of SMN and Gemin 3 and 4 proteins, as described previously (Cell. 1995; 80 (1): 155-65). Gemin 5 was also detected by some, but not all, sera that immunoprecipitated SMN, Gemin 3 and Gemin 4. Thus, Gemin 5 was not included to define the presence of anti-SMN complex antibodies.

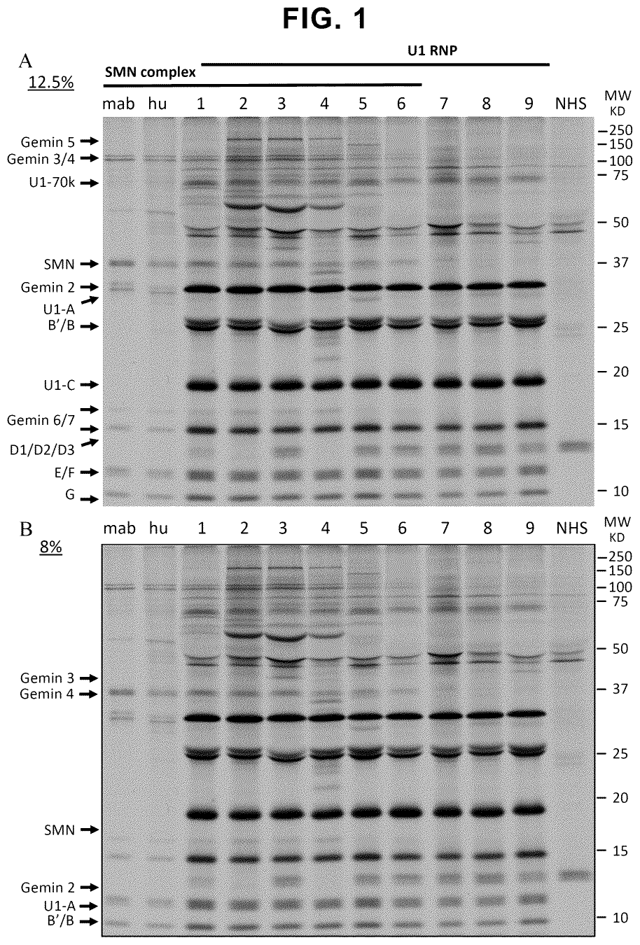

FIG. 1 shows immunoprecipitation using sera from the patients with MCTD. From the left, the immunoprecipitation patterns of anti-SMN mouse monoclonal antibodies, and human serum with monospecific anti-SMN complex antibodies. Lanes 1 to 9 are sera from the MCTD patients, and normal human serum. All sera from patients with MCTD showed strong immunoprecipitation of all components of U1 RNP, from U1-70 k to G proteins. In addition, sera in Lanes 1-6 demonstrated strong reactivity with components of SMN complex; SMN and Gemins 3 and 4 and in some cases Gemin 5 and considered positive for anti-SMN complex. In contrast, sera in Lanes 7-9 showed immunoprecipitation of U1 RNP, but not the SMN complex.

### (ELISA)

Antibodies to Ro52/TRIM21 were tested by enzyme-linked immunosorbent assay (ELISA) as described previously (Sci Rep. 2022; 12 (1): 11122). Ro52 recombinant proteins were purchased from Diarect GmbH (Freiburg, Germany). Briefly, 96-well microtiter plates (Immobilizer Amino; Nunc, Naperville, IL, USA) were coated with 0.5 μg/ml of recombinant protein and blocked with 0.5% bovine serum albumin (BSA)-NET/IGEPAL CA-630 for 1 hr at room temperature. Patients' sera (1:250) and alkaline phosphatase-conjugated goat anti-human IgG (1:1000; γ-chain specific; Jackson ImmunoResearch, West Grove, PA, USA) diluted in 0.5% BSA-NET/IGEPAL CA-630 were used as the sample and secondary antibodies, respectively. A standard curve was generated using serial 1:5 dilutions of a high-titer prototype serum. Optical density of samples measured at 405 nm was converted into units based on the standard curve.

### (Survival Rates)

Survival rates for 60 patients with MCTD were analyzed after 1 year of the intervention, excluding patients who were not followed-up after 1 year or were transferred to other hospitals (n=7). The cause of death was determined by reviewing the medical records.

### (Statistical Analysis)

Data are expressed as median (interquartile range) or number (%). Differences among groups were compared using Mann-Whitney U test, t-test, Fisher's exact test and one-way analysis of variance. For survival analysis, P-values were determined by Log-rank test. All reported P-values are two-sided and are not adjusted for multiple testing. The level of significance was set at P<0.05. All analyses were conducted using JMP version 11.0 software (SAS Institute Inc., Cary, NC, USA).

### 2. Results

### (Basic Clinical Characteristics)

Table 1 shows the basic clinical characteristics of patients with MCTD, SLE, and SSc.

TABLE 1

| | MCTD | | SLE | | SSc | |
|---|---|---|---|---|---|---|
| Variables | (n = 67) | | (n = 74) | | (n = 17) | |
| Mean age at onset (±SD) | 49.6 | (16.5) | 38.1 | (15.5) | 59.1 | (13.9) |
| Female, n (%) | 61 | (91.0) | 64 | (86.5) | 14 | (82.4) |
| Disease duration (years) | 0.7 | (0.4, 1.5) | 0.5 | (0.1, 1.7) | 1.0 | (0.5, 3.0) |
| Common manifestations, (%) | | | | | | |
| Raynaud's phenomenon | 100 | | 43.2 | | 100 | |
| Duration of Raynaud's phenomenon | 1.0 | (0.4, 3.3) | 2.0 | (0.8, 5.0) | 3.0 | (2 [symbol] 10) |

| | | | |
|---|---|---|---|
| Characteristic organ involvement, (%) | | | |
| Pulmonary arterial hypertension | 24.2 | 2.7 | 11.8 |
| Aseptic meningitis | 6.0 | 0.0 | 0.0 |
| SLE-like manifestations, (%) | | | |
| Polyarthritis | 71.6 | 56.8 | 17.6 |
| Lymphadenopathy | 44.8 | 44.6 | 5.9 |
| Facial erythema | 20.9 | 59.5 | 0.0 |
| Pericardial effusion or Pleural effusion | 10.4 | 20.3 | 5.9 |
| Cytopenia | 49.3 | 62.2 | 23.5 |
| CH50 <30 U/ml | 4.5 | 45.9 | 0.0 |
| SSc-like manifestations, (%) | | | |
| Sclerodactyly | 89.6 | 0.0 | 100.0 |
| mRRS >4 points | 0.0 | 0.0 | 76.4 |
| Interstitial lung disease | 61.2 | 6.6 | 94.1 |
| KL-6 (U/ml) >500 U/ml | 29.9 | 6.7 | 64.7 |
| IIM-like manifestations, (%) | | | |
| Muscle weakness | 13.4 | 5.4 | 0.0 |
| CK (U/l) >248 U/L | 32.8 | 2.7 | 0.0 |
| EMG changes | 4.5 | 0.0 | 0.0 |
| Other symptoms or involvement, (%) | | | |
| Fever | 26.9 | 43.2 | 0.0 |
| Nephritis | 0.0 | 44.6 | 0.0 |
| Other laboratory data, (%) | | | |
| CRP >0.3 mg/dl | 37.3 | 39.2 | 41.2 |
| ESR >1.5 mm/hr | 85.1 | 87.8 | 76.4 |
| IgG >1700 mg/dl | 77.6 | 64.9 | 41.2 |
| Nailfold video capillaroscopy, (%) | | | |
| Scleroderma patterns | 40.6 | 4.8 | 88.2 |
| Combinations of manifestations, (%) | | | |
| SLE like + SSc like | 64.2 | — | — |
| SLE like + IIM like | 8.9 | — | — |
| SSc like + IIM like | 1.5 | — | — |
| SLE like + SSc like + IIM like | 25.4 | — | — |

[symbol]

indicates data missing or illegible when filed

Unless otherwise noted, data are expressed as median (interquartile range) in Table 1. MCTD: mixed connective tissue disease, SLE: systemic lupus erythematosus, SSc: systemic sclerosis, SD: standard deviation, mRSS: modified Rodnan skin score, CK: creatine kinase, EMG: electromyogram, KL-6: krebs von den lungen-6, CRP: C-reactive protein, ESR: erythrocyte segmentation rate.

67 patients with MCTD, anti-U1 RNP antibodies-positive 74 patients with SLE and 17 patients with SSc participated in this study (Table 1). All patients participated in this study were newly diagnosed cases and had not received treatment with glucocorticoid (GC) or immunosuppressants. The mean age of the patients with MCTD was 49.6 years and most of them were women (91.0%). The duration of disease (median) was 0.7 years, all patients had Raynaud's phenomenon, 24.2% of the patients had PAH, 49.3% had cytopenia, 61.2% had ILD and 13.4% had muscle weakness. When the disease types were classified based on the combination of organ involvements, the SLE-like+SSc-like type was the most common (64.2%), followed by the SLE-like+SSc-like+IIM-like type (25.4%).

Compared with patients with SLE, patients with MCTD were more likely to have Raynaud's phenomenon and less likely to have facial erythema, cytopenia, nephritis and hypocomplementemia. In addition, compared with patients with SSc, patients with MCTD were more likely to have fever or hypergammaglobulinemia, with no difference in the frequence of Raynaud's phenomenon.

### (Prevalence of Anti-SMN Complex Antibodies in MCTD)

Table 2 shows the prevalence of anti-SMN complex antibodies in patients with MCTD, SLE and SSc.

TABLE 2

| | MCTD | SLE | SSc | P-value | |
|---|---|---|---|---|---|
| Autoantibodies, (%) | (n = 67) | (n = 74) | (n = 17) | (vs. SLE) | (vs. SSc) |
| Anti-SMN complex Abs, n(%) | 24 (35.8) | 6 (8.1) | 2 (11.8) | <0.001 | 0.078 |
| Anti-U1 RNP Abs - high level | 88.1 | 43.2 | 41.2 | <0.001 | <0.001 |
| (≥550 U/ml) | | | | | |
| Anti-Sm Abs | 19.4 | 60.8 | 17.6 | <0.001 | 1.0 |
| Anti-ds DNA Abs | 1.5 | 56.8 | 0.0 | <0.001 | 0.12 |
| Anti-centromere Abs | 6.0 | 2.7 | 23.5 | 0.43 | 0.049 |
| Anti-Scl-70 Abs | 0.0 | 0.0 | 11.8 | — | 0.039 |
| Anti-RNA polymerase III Abs | 0.0 | 2.7 | 0.0 | 0.50 | — |
| Anti-ARS Abs | 1.5 | 0.0 | 0.0 | 0.48 | 1.0 |
| Anti-SS-A/Ro60 Abs | 46.3 | 68.9 | 23.5 | 0.010 | 0.11 |
| Anti-Ro52 Abs | 36.4 | 63.5 | 35.3 | 0.002 | 1.0 |
| Anti-SS-B Abs | 9.0 | 20.3 | 5.9 | 0.10 | 1.0 |

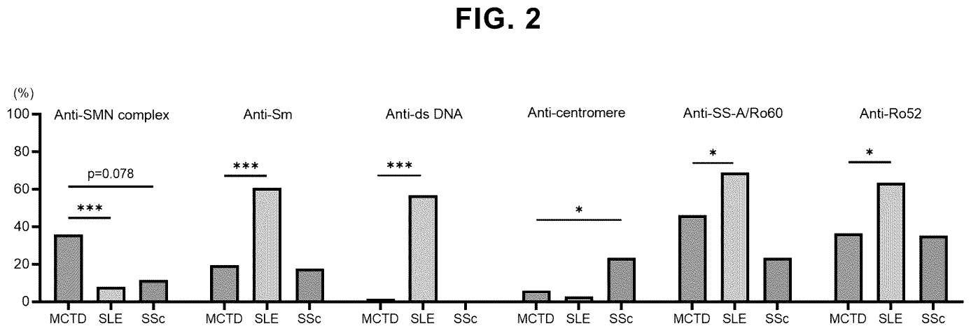

The prevalence of anti-SMN complex antibodies in patients with MCTD was 35.8% (24/67 patients) (FIG. 2, Table 2). This was higher than those in patients with SLE (8.1%, 6/74) and SSc (11.8%, 2/17) (P<0.001 and P=0.078, respectively). The prevalences of other autoantibodies were as follows. Anti-U1 RNP antibodies were positive in all MCTD patients, and even when limited to those with high anti-U1 RNP antibodies titer (≥5500 U/ml), they were found in 88.1% of the patients. On the other hand, the anti-U1 RNP antibodies were detected in Anti-U1 RNP antibodies were also found in half or more of SLE and SSc patients (SLE: 56.2%, SSc: 52.9%). In addition, SLE-specific autoantibodies, anti-Sm antibodies (MCTD: 19.4%, SLE: 60.8%) and anti-dsDNA antibodies (MCTD: 1.5%, SLE: 56.8%), were more prevalent in patients with SLE, and copresence of systemic sclerosis-specific autoantibodies, anti-centromere antibodies (MCTD: 6.0%, SSc: 23.5%) and anti-Scl-70 antibodies (MCTD: 0.0%, SSc: 11.8%), were more prevalent in patients with SSc.

Sensitivity, specificity and positive/negative prevalence of each autoantibody for MCTD diagnosis were calculated in this study (Table 3).

TABLE 3

| | Sensitivity | Specificity | PPV | NPV |
|---|---|---|---|---|
| | (%) | (%) | (%) | (%) |
| Anti-U1 RNP Abs - high | 88.1 | 57.1 | 60.2 | 86.7 |
| level (≥550 U/ml) | | | | |
| Anti-SMN complex Abs | 35.8 | 91.2 | 75.0 | 66.0 |
| Anti-SS-A/Ro60 Abs | 46.3 | 50.5 | 40.8 | 56.1 |
| Anti-Ro52 Abs | 35.8 | 41.8 | 31.2 | 46.9 |
| Anti-SS-B/La Abs | 9.0 | 85.7 | 31.6 | 56.1 |

First, the sensitivity of anti-SMN complex antibodies was lower than that of anti-U1 RNP antibodies (100%) and anti-U1 RNP antibodies high level positive (92.5%). On the other hand, with regards to specificity and positive predictive value, as compared to specificity of 37.0% and positive predictive value of 56.0% (65/116) for anti-U1 RNP antibodies high level positive, anti-SMN complex antibodies shows higher specificity of 91.2% and positive predictive value of 75.0% (24/32), respectively. These findings suggest that the presence of anti-SMN complex antibodies is highly specific to MCTD relatively.

### (Association Between Anti-SMN Complex Antibodies and Clinical/Laboratory Findings in MCTD)

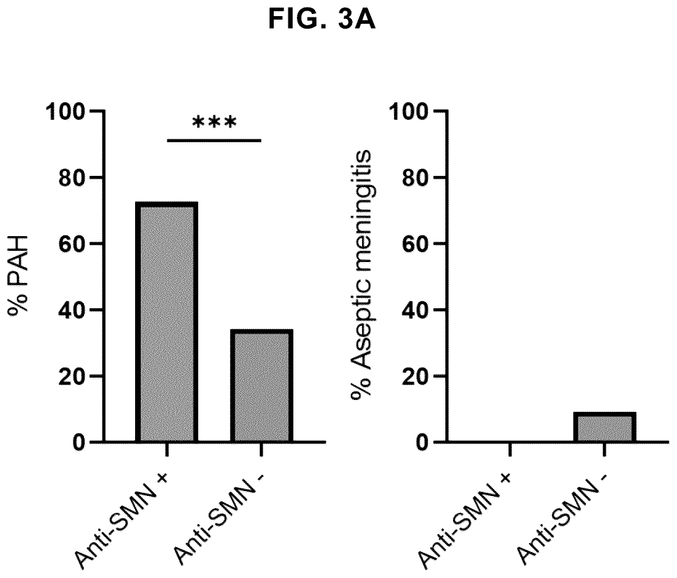

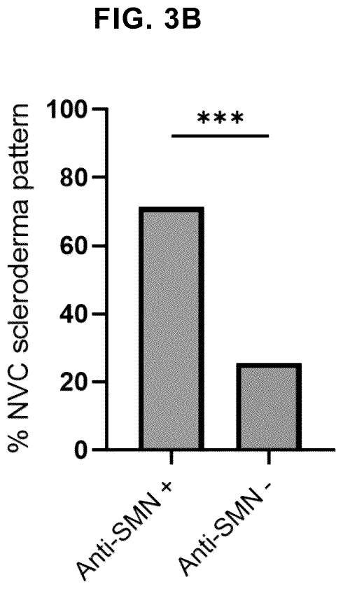

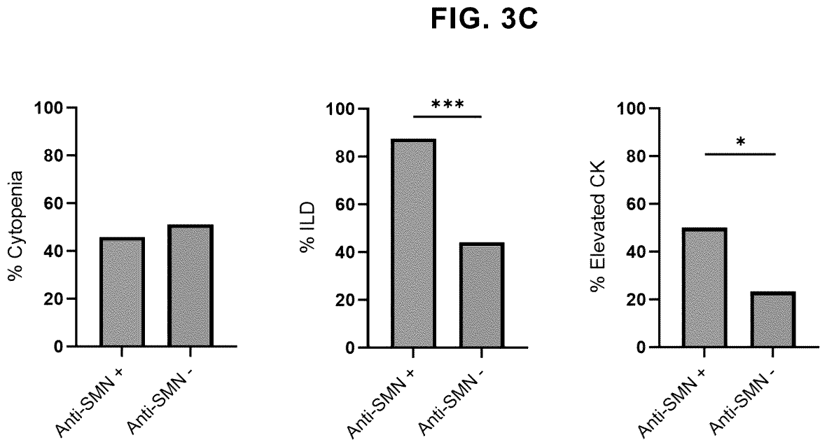

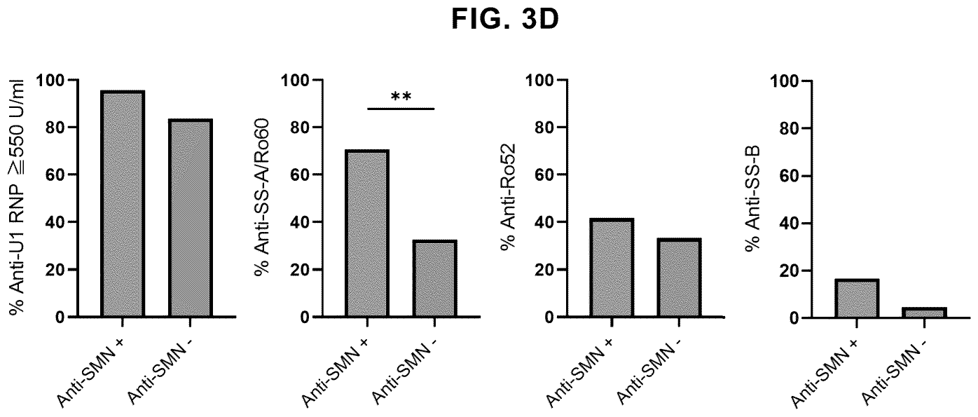

The clinical significance of anti-SMN complex antibodies was then examined. The clinical characteristics of anti-SMN complex antibodies-positive patients (n=23) and anti-SMN complex antibodies-negative patients (n=44) are summarized in FIG. 3 and Table 4.

TABLE 4

| | Anti-SMN complex antibodies status | | | |
|---|---|---|---|---|
| | Positive | Negative | | Odds ratio |
| Variables | (n = 24) | (n = 43) | P-value | (95% CI) |
| Mean age at onset (±SD) | 58.5 (16.0) | 44.7 (14.9) | <0.001 | |
| Female, n (%) | 21 (91.7) | 39 (90.7) | 1.00 | |
| Disease duration (years) | 1.1 (0.5, 1.7) | 0.7 (0.4, 1.5) | 0.39 | |
| Characteristic organ | | | | |
| involvement, (%) | | | | |
| Pulmonary arterial | 54.2 | 7.0 | <0.001 | 15.7 (3.8-65.3) |
| hypertension | | | | |
| BNP >40 pg/dl | 68.2 | 21.9 | 0.002 | 7.7 (2.2-26.1) |
| TR-PG >25 mmHg | 60.9 | 9.5 | <0.001 | 15.8 (4.2-59.3) |
| Aseptic meningitis | 0.0 | 9.3 | 0.29 | |
| SLE-like manifestations, (%) | | | | |
| Polyarthritis | 75.0 | 69.8 | 0.78 | |
| Lymphadenopathy | 39.1 | 46.5 | 0.80 | |
| Facial erythema | 17.4 | 23.3 | 0.76 | |
| Pericardial effusion or | 17.4 | 7.0 | 0.22 | |
| Pleural effusion | | | | |
| Cytopenia | 45.8 | 51.2 | 0.61 | |
| SSc-like manifestations, (%) | | | | |
| Sclerodactyly | 95.8 | 86.0 | 0.41 | |
| Interstitial lung disease | 87.5 | 44.2 | <0.001 | 8.8 (2.2-34.1) |
| KL-6 > [symbol] 00 U/ml | 45.8 | 21.4 | 0.051 | 3.6 (1.2-10.7) |
| IIM-like manifestations, (%) | | | | |
| Muscle weakness | 16.7 | 11.7 | 0.71 | |
| CK >248 U/L | 50.0 | 23.3 | 0.033 | 3.3 (1.1-9.6) |
| Other symptoms or | | | | |
| involvement, (%) | | | | |
| Fever | 16.7 | 31.8 | 0.25 | |
| Dyspnea | 60.9 | 20.4 | 0.003 | 5.3 (1.8-15.8) |
| Other laboratory findings, (%) | | | | |
| CRP >0.3 mg/dl | 41.7 | 34.9 | 0.61 | |
| ESR >1.5 mm/hr | 95.8 | 79.1 | 0.082 | |
| IgG >1700 mg/dl | 91.3 | 69.8 | 0.065 | |
| CH50 <30 U/ml | 8.3 | 2.3 | 0.29 | |
| Autoantibodies, (%) | | | | |
| Anti-U1 RNP Abs - high | 95.8 | 83.7 | 0.24 | |
| level (≥550 U/ml) | | | | |
| Anti-Sm Abs | 25.0 | 16.3 | 0.52 | |
| Anti-SS-A/Ro60 Abs | 70.8 | 32.6 | 0.005 | 5.0 (1.7-14.9) |
| Anti-Ro52 Abs | 41.7 | 33.3 | 0.00 | |
| Anti-SS-B Abs | 16.7 | 4.7 | 0.18 | |
| Pulmonary function test | | | | |
| % FVC <80% | 60.0 | 25.8 | 0.02 | 4.3 (1.3-14.3) |
| % DLCO <80% | 84.2 | 51.5 | 0.03 | 5.0 (1.2-20.5) |
| Nailfold video capillaroscopy | | | | |
| Scleroderma pattern (%) | 71.4 | 25.6 | <0.001 | 7.3 (2.3-23.4) |
| Initial Treatment, (%) | | | | |
| High-dose glucocorticoid | 75.0 | 34.9 | 0.005 | 5.0 (1.7-14.9) |
| therapy | | | | |
| Intravenous | 37.5 | 14.0 | 0.035 | 3.7 (1.1-12.2) |
| cyclophosphamide | | | | |

[symbol]

indicates data missing or illegible when filed

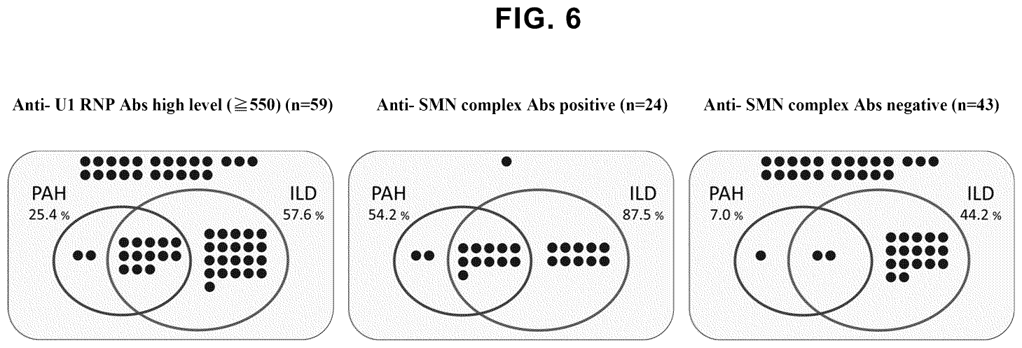

Anti-SMN complex antibodies-positive patients showed characterized by a higher prevalence of PAH complications (54.2% vs 7.0%, P<0.001, OR 15.7), nailfold capillary abnormalities (71.4% vs 25.6%, P<0.001) and ILD complications (87.5% vs 44.2%, P=0.001, OR 8.8 W). In fact, patients positive for anti-SMN complex antibodies had a higher prevalence of either PAH or ILD complications compared with patients with high levels of anti-U1 RNP antibodies positive, and 95.8% (23/24) were complicated with PAH or ILD (FIG. 6). With regard to the relationship with other autoantibodies, anti-SMN complex antibodies-positive patients showed a significantly higher prevalence of anti-SS-A/Ro60 antibodies (70.8% vs 32.6%, P=0.01).

In MCTD, SLE-like, sclerosis-like, and IIM-like findings are mixed. When disease types were classified based on the combination of overlapping manifestations, a half or more (54.2%) of the anti-SMN complex antibodies-positive patients had been classified to a disease type having all features of SLE-like, sclerosis-like and IIM-like findings.

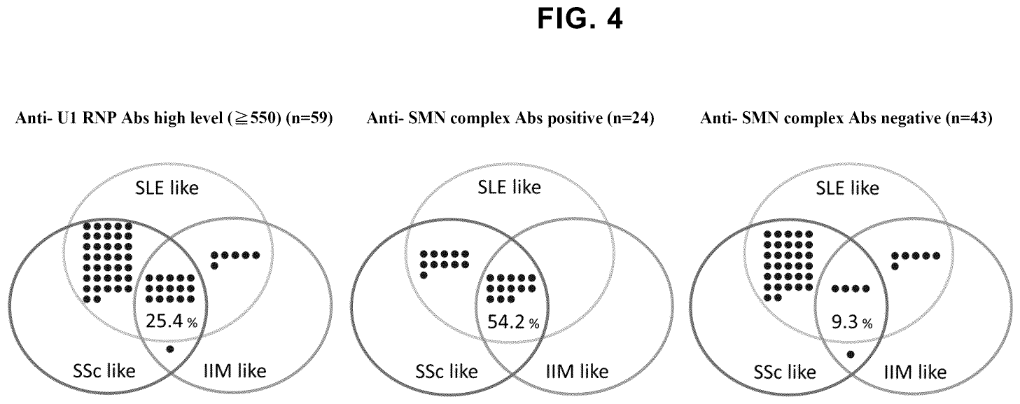

Conversely, the proportion of such patients was low in patients with high levels of anti-U1 RNP antibodies positive and anti-SMN complex antibodies-negative patients (25.4% and 9.3%, respectively) (FIG. 4).

(Prognosis of MCTD Patients with Anti-SMN Complex Antibodies)

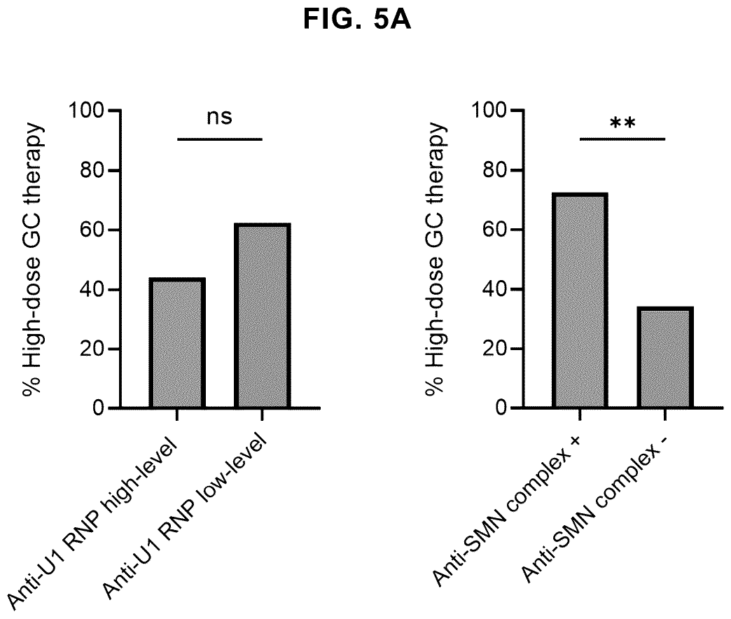

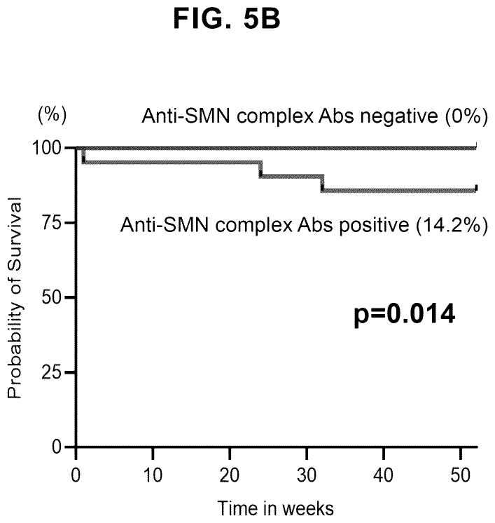

Finally, the prognosis of patients (n=60) who were followed for at least 1 year by August 2022 was compared (FIG. 5). In terms of initial treatment, the high percentage of anti-SMN complex antibodies-positive patients received high-dose glucocorticoid therapy (72.7% vs. 34.2%, p=0.007) (FIG. 5A). Regarding the survival rate after 1 year, all of negative patients were alive, whereas three of anti-SMN complex antibodies-positive patients died, resulting in a lower survival rate among antibodies-positive patients (14.2% vs. 0.0%, p=0.014, FIG. 5B). The cause of death was exacerbation of PAH or heart failure in two patients and severe infection associated with immunosuppressive therapy in one patient. These results suggest that anti-SMN complex antibodies-positive MCTD has a poor prognosis.

### 3. Discussion

Anti-U1 RNP antibody is an autoantibody essential for the diagnosis of MCTD, however, anti-U1 RNP antibody-positive patients exist in SLE and sclerosis and the disease specificity is not high. In the present study, autoantibody analysis was performed in new-onset MCTD, and the three major clinical significances of anti-SMN complex antibodies in MCTD were found as follows.

A clinically important point is that anti-SMN complex antibodies-positive MCTD has a poorer prognosis than negative cases. This may be due to the high rate of complications with PAH and ILD, which are poor prognostic factors in MCTD (J Rheumatol. 2013; 40 (7): 1134-42). Although anti-U1 RNP antibodies are known to be associated with PAH in autoimmune diseases, this study suggested that coexisting anti-SMN complex antibodies, rather than anti-U1 RNP antibodies, may be a biomarker for severe PAH and ILD directly related to the prognosis (Arthritis Rheumatol. 2016; 68 (2): 484-93).

The second point relates to diagnosis. Anti-SMN complex antibodies were found more frequently in MCTD than in SLE or SSc. Although this study was conducted only on anti-U1 RNP antibody-positive patients and did not examine anti-U1 RNP antibody-negative patients, the presence of anti-SMN complex antibody in the presence of anti-U1 RNP antibody highly suggests the presence of MCTD. In other words, anti-SMN complex antibodies may assist the diagnosis by compensating for the low specificity of anti-U1 RNP antibodies in the diagnosis of MCTD and may express the disease state.

In addition, anti-SMN complex antibodies-positive patients were shown to be frequently found in a disease type with all clinical features of SLE-, SSc- and IIM findings. In other words, anti-SMN complex antibody-positive MCTD is associated with ILD and PAH and can be considered as a more typical MCTD (‘particular’ MCTD). These results support the point that the disease concept of MCTD is not simply anti-U1 RNP antibody disease, and that characteristic clinical findings, such as those exhibited by anti-SMN complex antibodies-positive MCTD, are important.

In a previous report on other autoantibodies in MCTD, anti-Ro52 antibodies were reported to be associated with activity of ILD in MCTD (Rheumatology (Oxford). 2018; 57 (2): 255-62, Rheumatology (Oxford). 2016; 55 (1): 103-8). However, in the present study, comprehensive analysis using immunoprecipitation demonstrated that anti-SMN complex antibodies are more significant for the MCTD disease entity than the high levels of anti-U1 RNP antibodies positive or anti-Ro52 antibodies.

On the other hand, the pathogenicity of the SMN complex (SMN+Gemin 2-7) itself is unknown. In eukaryotic cells, the SMN complex plays a key role in the assembly of snRNPs (U1 RNP, Sm) (Arthritis Rheum. 2011; 63 (7): 1972-8, Clin Rev Allergy Immunol. 2012; 42 (1): 16-25). However, the mechanism of production and function of this antibody needs to be elucidated.

This study is the first report of the clinical significance of anti-SMN complex antibodies in MCTD patients. This study suggests that anti-SMN complex antibodies are relatively specific to MCTD and may be a novel biomarker for PAH/ILD complications, which are poor prognostic factors. The results of this study may help in the application of sub-grouping and personalized medicine in existing MCTD.

### INDUSTRIAL APPLICABILITY

The present invention provides a new biomarker for evaluating severity or prognosis of MCTD, and makes it possible to evaluate severity, organ involvement, disease activity, and prognosis of MCTD. Although the severity classification is used for the medical expense benefit for MHLW-designated intractable diseases, it is composed mostly of subjective items. The present invention enables severity classification of MCTD based on objective indexes.

1. A method for evaluating severity of a patient with mixed connective tissue disease, which comprises
1) a step of bringing a biological sample separated from a patient with mixed connective tissue disease into contact with a carrier in which an antigen containing one or more of component proteins of survival motor neuron complex or an antigenic partial peptide thereof is immobilized on a solid phase to form a complex of an autoantibody in the biological sample and the antigen immobilized on the solid phase,
2) a step of detecting the autoantibody in the complex of the autoantibody and the immobilized antigen, and
3) a step of associating the patient with a severe mixed connective tissue disease when the autoantibody is detected from the biological sample separated from the patient in step 2).

2. The method of claim 1, wherein the component protein of the survival motor neuron complex is selected from the group consisting of SMN, Gemin 3, Gemin 4 and Gemin 5.

3. The method of claim 1, wherein the detection of the autoantibody is performed by bringing an anti-human IgG antibody or a functional binding fragment thereof into contact with the complex of the autoantibody and the immobilized antigen.

4. The method of claim 1, wherein the severe mixed connective tissue disease is a mixed connective tissue disease complicated by pulmonary arterial hypertension or interstitial lung disease.

5. The method of any of claim 1, wherein the severe mixed connective tissue disease is a mixed connective tissue disease accompanied by all of systemic lupus erythematosus-like, systemic sclerosis-like, and idiopathic inflammatory myopathy-like clinical symptoms.

6. A method for evaluating prognosis of a patient with mixed connective tissue disease, which comprises
1) a step of bringing a biological sample separated from a patient with mixed connective tissue disease into contact with a carrier in which an antigen containing one or more of component proteins of survival motor neuron complex or an antigenic partial peptide thereof is immobilized on a solid phase to form a complex of an autoantibody in the biological sample and the antigen immobilized on the solid phase,
2) a step of detecting the autoantibody in the complex of the autoantibody and the immobilized antigen, and
3) a step of associating the patient with a mixed connective tissue disease with a poor prognosis when the autoantibody is detected from the biological sample separated from the patient in step 2).

7. The method of claim 6, wherein the component protein of the survival motor neuron complex is selected from the group consisting of SMN, Gemin 3, Gemin 4 and Gemin 5.

8. The method of claim 6, wherein the detection of the autoantibody is performed by bringing an anti-human IgG antibody or a functional binding fragment thereof into contact with the complex of the autoantibody and the immobilized antigen.

9. A method of treating mixed connective tissue disease, which comprises detecting an autoantibody to survival motor neuron complex or a component protein of the survival motor neuron complex in a biological sample separated from a patient with mixed connective tissue disease, and administering glucocorticoid to the patient with the mixed connective tissue disease when the patient is positive for the autoantibody against the survival motor neuron complex or the component protein of the survival motor neuron complex.

10. The method of claim 9, wherein the detection of an autoantibody against survival motor neuron complex or a component protein of the survival motor neuron complex is performed by a method comprising
1) a step of bringing a biological sample separated from a patient with mixed connective tissue disease into contact with a carrier in which an antigen containing one or more of component proteins of the survival motor neuron complex or an antigenic partial peptide thereof is immobilized on a solid phase to form a complex of an autoantibody in the biological sample and the antigen immobilized on the solid phase, and
2) a step of detecting the autoantibody in the complex of the autoantibody and the immobilized antigen.

11. The method of claim 10, wherein the component protein of the survival motor neuron complex is selected from the group consisting of SMN, Gemin 3, Gemin 4 and Gemin 5.

12. The method of claim 10, wherein the detection of the autoantibody is performed by bringing an anti-human IgG antibody or a functional binding fragment thereof into contact with the complex of the autoantibody and the immobilized antigen.
